(19) United States
(12) Patent Application Publication
(10) Pub. No.: US 2026/0283622 A1
(43) Pub. Date: Sep. 24, 2026

(54) Medical Device for Closing a Vessel

(71) Applicant: Bard Peripheral Vascular, Inc., Franklin Lakes, NJ (US)

(72) Inventors: Andrzej J. Chanduszko, Chandler, AZ (US); Alexander G. Lastovich, Gilbert, AZ (US)

(21) Appl. No.: 19/479,217

(22) PCT Filed: Apr. 28, 2023
(86) PCT No.: PCT/US2023/020319

§ 371 Date: Oct. 27, 2025

(51) Int. Cl. A61B 17/12; A61B 17/00

(52) U.S. Cl. CPC A61B 17/12186; A61B 17/00234; A61B 17/12031; A61B 17/12109; A61B 2017/00305; A61B 2017/00544; A61B 2017/00561; A61B 2017/00778; A61B 2017/00862; A61B 2017/00867; A61B 2017/00951

(57) ABSTRACT

A medical device for closing a vessel. The medical device comprises a catheter shaft defining a longitudinal axis, and an expansion element positioned at or near a distal end of the catheter shaft. The expansion element has a radially contracted configuration and a radially expanded configuration. The expansion element is configured to flatten the vessel in the radially expanded configuration. The medical device further comprises one or more injection ports for injecting an adhesive into the vessel.

### TECHNICAL FIELD

The present disclosure relates to a medical device for closing a vessel, and a method of closing a vessel using a medical device.

### BACKGROUND

Chronic Venous Insufficiency (CVI) is a medical condition which occurs when the valves in a vein become damaged allowing blood to leak backwards thereby causing pooling of blood in the veins and elevated blood pressure. Such valve damage may occur as the result of aging, extended sitting or standing, or a combination of aging and reduced mobility.

Treatments for CVI include surgically removing or repairing the veins, laser or RF ablation, and sclerotherapy where a sclerosant is injected into the veins to make them shrink. However, surgical treatments are complex and time intensive and can result in long recovery times for the patient. Furthermore, laser or RF ablation and sclerotherapy have a risk of causing nerve damage and often require the use of tumescent anesthesia thereby reducing patient comfort.

In some cases, CVI is also treated by injecting an adhesive into the vein to seal or close the vein, thereby rerouting blood to nearby healthy veins. However, this treatment requires manual compression of the vein after the injection of the adhesive in order to seal the vein. This treatment therefore requires the presence of an additional medical practitioner and furthermore makes this treatment only suitable for surface level veins.

There is hence a need in the art for a new type of medical device for closing a blood vessel, and specifically a vein, in a safe, effective and quick manner using non-tumescent, non-thermal and non-sclerosant means.

### SUMMARY

In a first aspect of the present disclosure, there is provided a medical device for closing a vessel. The medical device comprises a catheter shaft defining a longitudinal axis, and an expansion element positioned at or near a distal end of the catheter shaft. The expansion element has a radially contracted configuration and a radially expanded configuration. The expansion element is configured to flatten the vessel in the radially expanded configuration. The medical device further comprises one or more injection ports for injecting an adhesive into the vessel.

In some embodiments, this may result in a medical device which can safely and effectively close a vessel using non-tumescent, non-thermal and non-sclerosant means and avoids the requirement for manual vein compression.

The vessel may specifically be a blood vessel such as a vein.

The medical device may further comprise one or more suction ports for reducing the pressure in the vessel.

In some embodiments, this may help to aspirate blood and flatten the vessel thereby resulting in more effective closure of the vessel using less adhesive.

The one or more suction ports may be positioned on the catheter shaft.

At least one of the one or more suction ports may be positioned at the distal end of the catheter shaft.

In some embodiments, this may result in more effective flattening of the vessel.

The one or more suction ports may be positioned distally of the expansion element.

In some embodiments, this may result in more effective flattening of the vessel.

The one or more suction ports may be positioned distally of the one or more injection ports.

In some embodiments, this may help to reduce the amount of adhesive required to close the vessel.

The medical device may further comprise a vacuum pump connected to the one or more suction ports through a suction lumen.

The one or more injection ports may be positioned at the same longitudinal position as the expansion element.

In some embodiments, this may help to reduce the amount of adhesive required to effectively close the vessel.

At least one of the one or more injection ports may be positioned on the catheter shaft.

In some embodiments, this may result in effective injection of adhesive from the medical device into the vessel.

The medical device may comprise a plurality of injection ports arranged along the longitudinal axis.

In some embodiments, this may result in effective injection of adhesive from the medical device into the vessel.

A distal injection port of the plurality of injection ports may have a greater opening than a proximal injection port of the plurality of injection ports.

The more distally positioned injection ports may have a greater diameter than the more proximally positioned injection ports.

In some embodiments, this may result in a more even distribution of adhesive.

The catheter shaft may comprise an injection lumen for supplying an adhesive to the one or more injection ports.

The medical device may further comprise an adhesive pump connected to the one or more injection ports via the injection lumen.

The expansion element may move between the radially contracted configuration and the radially expanded configuration in a plane coplanar with the longitudinal axis.

In some embodiments, this may result in more effective flattening of the vessel when moving the expansion element from the radially contracted configuration to the radially expanded configuration.

The expansion element may comprise a first expansion member and a second expansion member positioned on opposite sides of the catheter shaft.

In some embodiments, this may result in more effective flattening of the vessel when moving the expansion element from the radially contracted configuration to the radially expanded configuration.

The first expansion member and the second expansion member may have a rough or serrated surface.

In some embodiments, this may additionally cause mechanical irritation of the vessel wall resulting in vessel closure.

The first expansion member and the second expansion member may be flexible and may be configured to bend radially outwards in the radially expanded configuration.

In some embodiments, this may allow for simple and effective movement of the first expansion member and the second expansion member between the radially contracted configuration and the radially expanded configuration.

The first expansion member and the second expansion member may have a convex shape in the radially expanded configuration.

The first expansion member may comprise a first tube and the second expansion member may comprise a second tube.

At least one of the one or more injection ports may be positioned on the first tube and/or the second tube.

In some embodiments, this may result in effective distribution of adhesive in the vessel.

The at least one of the one or more injection ports may be positioned on the radial inside surface of the first tube and/or the second tube.

In some embodiments, this may result in effective distribution of adhesive in the vessel.

The first expansion member may comprise a wire or ribbon and the second expansion member may comprise a wire or ribbon.

In some embodiments, this may allow for a simpler construction of the device.

The one or more injection ports may be positioned on the catheter shaft.

The expansion element may be made from a metal or metal alloy.

The expansion element may be made from a superelastic material.

In some embodiments, this may further radial expansion of the expansion element thereby resulting in more effective flattening of the vessel.

The expansion element may be made from a shape-memory material.

In some embodiments, this may allow the expansion element to move from the radially contracted configuration to the radially expanded configuration when introduced into a vessel.

The expansion element may be made from Nitinol.

The catheter shaft may comprise an outer shaft and an inner shaft.

The inner shaft may be longitudinally moveable relative to the outer shaft.

A proximal end of the expansion element may be connected to the outer shaft and a distal end of the expansion element may be connected to the inner shaft such that longitudinal movement of the inner shaft relative to the outer shaft moves the expansion element between the radially contracted configuration and the radially expanded configuration.

In some embodiments, this may allow for controlled and effective movement of the expansion element between the radially contracted configuration and the radially expanded configuration.

The catheter shaft may further comprise a guidewire lumen for accommodating a guidewire.

In a second aspect of the present disclosure there is provided, a medical device for closing a vessel. The medical device comprises a catheter shaft defining a longitudinal axis; one or more flexible tubes positioned at or near a distal end of the catheter shaft, the expansion element having a radially contracted configuration and a radially expanded configuration, wherein the expansion element is configured to flatten the vessel in the radially expanded configuration; and one or more injection ports associated with the one or more flexible tubes or the catheter shaft for injecting an adhesive into the vessel.

In a third aspect of the present disclosure, there is provided a medical device for closing a vessel. The medical device comprises a catheter shaft defining a longitudinal axis, the catheter shaft including means for flattening the vessel and injecting an adhesive therein.

The means for flattening and injecting may comprise one or more tubes or wires positioned at or near a distal end of the catheter shaft and having a radially contracted configuration and a radially expanded configuration to flatten the vessel, and further including one or more injection ports for injecting an adhesive into the vessel.

In a fourth aspect of the present disclosure, there is provided a method of closing a vessel using a medical device having a catheter shaft, an expansion element and one or more injection ports. The method comprises inserting the medical device into a vessel; flattening the vessel by moving the expansion element from a radially contracted configuration to a radially expanded configuration; and injecting an adhesive into the vessel through the one or more injection ports.

The method may further comprise reducing the pressure in the vessel through one or more suction ports of the medical device.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a medical device for closing a vessel according to one or more embodiments of the present disclosure having an expansion element in a radially contracted configuration;

FIG. 2A shows a distal end of the medical device of FIG. 1 with the expansion element in a radially expanded configuration;

FIGS. 2B and 2C show cross-sectional views of the medical device of FIG. 2A;

FIG. 3A shows the medical device of FIG. 1 being inserted into a vessel with the expansion element in the radially contracted configuration;

FIG. 3B shows the medical device of FIG. 1 in the vessel with the expansion element in the radially expanded configuration;

FIG. 4 shows a medical device for closing a vessel according to one or more alternative embodiments of the present disclosure; and

FIG. 5 shows a medical device for closing a vessel according to one or more alternative embodiments of the present disclosure; and

FIG. 6 shows a medical device for closing a vessel according to one or more alternative embodiments of the present disclosure.

### DETAILED DESCRIPTION

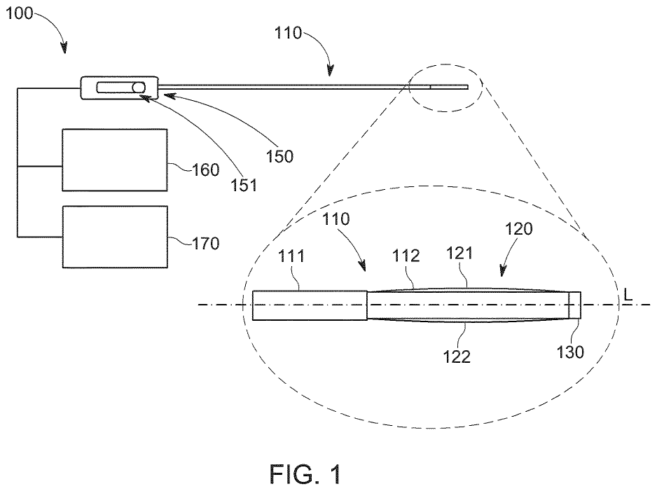

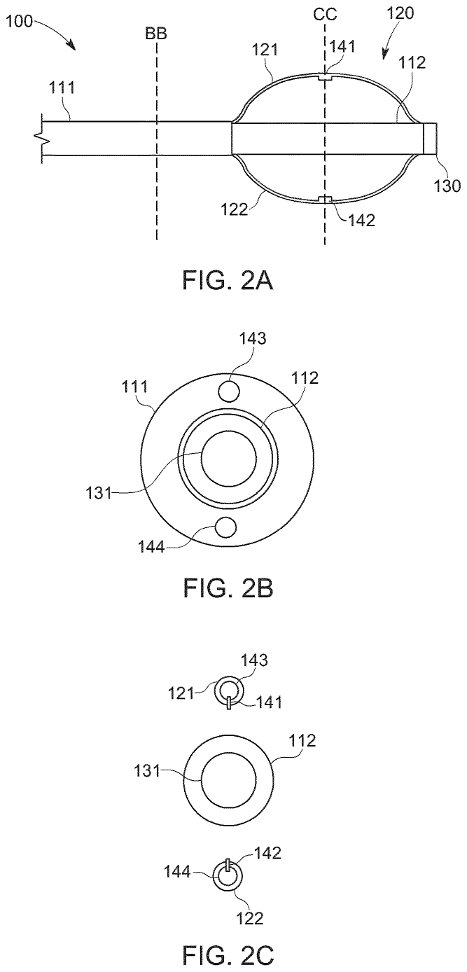

FIG. 1 shows a medical device 100 for closing a vessel, such as a vein. The medical device 100 comprises a catheter shaft 110 defining a longitudinal axis L. An expansion element 120 is positioned near a distal end of the catheter shaft 110. The expansion element 120 has a radially contracted configuration and a radially expanded configuration. In the radially expanded configuration, the radial extent of the expansion element 120 is greater than in the radially contracted configuration. FIG. 1 shows the expansion element in the radially contracted configuration. When inserted into a vessel, the expansion element 120 is configured to flatten the vessel when positioned in the radially expanded configuration. The medical device 100 further comprises one or more injection ports, such as first injection port 141 and second injection port 142 (see FIG. 2A), for injecting an adhesive into the vessel and thereby close the vessel by sealing it.

The catheter shaft 110 may comprise an outer shaft 111 and an inner shaft 112, with the inner shaft positioned within and longitudinally moveable relative to the outer shaft 111. The distal end of the inner shaft 112 may extend past the distal end of the outer shaft 111. The expansion element 120 may comprise a first expansion member 121 and a second expansion member 122 positioned on opposite sides of the catheter shaft 110. In the embodiment of FIG. 1, the first and second expansion members 121, 122 may be flexible tubes which can bend radially outwards when moving from the radially contracted configuration to the radially expanded configuration. A proximal end of the first and second expansion member 121, 122 may be connected to the distal end of the outer shaft 111 whilst a distal end of the first and second expansion member 121, 122 may be connected to the inner shaft 112. Longitudinal movement of the inner shaft 112 relative to the outer shaft 111 can therefore move the first and second expansion members 121, 122 between the radially contracted configuration and the radially expanded configuration. For example, proximal movement of the inner shaft 112 relative to the outer shaft 111 may move the expansion element 120, including the first and second expansion members 121, 122, from the radially contracted configuration to the radially expanded configuration (shown in FIG. 2A). Conversely, distal movement of the inner shaft 112 relative to the outer shaft 111 may move the expansion element 120, including the first and second expansion member 121, 122, from the radially expanded configuration (shown in FIG. 2A) to the radially contracted configuration shown in FIG. 1.

The first expansion member 121 and the second expansion member 122 may be made from a number of suitable materials which provide sufficient flexibility to allow the first and second expansion member to move between the radially contracted configuration and the radially expanded configuration. For example, the first expansion member 121 and the second expansion member 122 may be made from a superelastic material such as nitinol, other metals such as stainless steel or a CoCr alloy, or non-metallic materials such as polymers. The inner catheter shaft 112 and the outer catheter shaft 111 may be made from standard materials such as, for example, PEBAX, polyurethane, polyimide or combinations thereof. The first expansion member 121 and the second expansion member 122 may further have a rough or serrated abrasive surface such that they mechanically irritate the vessel wall when coming into contact with the vessel wall.

The medical device 100 may further comprise a suction port 130. The suction port 130 may be disposed at a distal end of the inner shaft 112, distally of the expansion element 120 and the first and second injection ports 141, 142 (shown in FIG. 2A). The suction port 130 may be connected to a vacuum pump 160 via a suction lumen 131 (shown in FIGS. 2B and 2C). When the vacuum pump 170 is turned on, the suction port 130 reduces the pressure in the blood vessel thereby helping to aspirate blood and flatten the vessel which results in more effective closure of the vessel using less adhesive.

The first injection port 141 (shown in FIG. 2A) may be positioned on the first expansion member 121 and may be connected to an adhesive pump 160 via a first injection lumen 143 (shown in FIGS. 2B and 2C). A second injection port 142 (shown in FIG. 2A) may be positioned on the second expansion member 122 and connected to the adhesive pump via a second injection lumen 144 (shown in FIGS. 2B and 2C). When the adhesive pump 160 is turned on an adhesive may be injected into the vessel through the injection ports 141, 142 in order to seal the vessel and close it. The adhesive may be any suitable tissue adhesive and may include, for example, cyanoacrylates or microbial transglutaminase (mTG) in liquid or gel form.

The medical device 100 may further include a handle 150 connected at a proximal end of the catheter shaft 110. The handle 150 may have an expansion control element 151, for example, in the form of a slider, as shown in FIG. 1. The expansion control element 151 may be connected to the inner shaft 112 to allow the inner shaft 112 to be moved longitudinally relative to the outer shaft 111 and thereby move the expansion element 120 between the radially contracted configuration and the radially expanded configuration. For example, proximal movement of the expansion control element 151 may move the inner shaft 112 proximally relative to the outer shaft 111 and thereby move the expansion element 120 from the radially contracted configuration to the radially expanded configuration. Similarly, distal movement of the expansion control element 151 may move the inner shaft 112 distally relative to the outer shaft 111 and thereby move the expansion element 120 from the radially expanded configuration to the radially contracted configuration. The expansion control element 151 may further have a locking feature which allows the expansion element 120 to be locked in the radially expanded configuration. The handle 150 may also connect the vacuum pump 160 and adhesive pump 170 to the catheter shaft 110.

FIG. 2A shows a side view of a distal section of medical device 100 with the expandable element 120 in the radially expanded configuration. In the radially expanded configuration, the first expandable member 121 and the second expandable member 122 may be bent radially outwards and take on a convex shape. The radial extent of the expandable element 120 is greater in the radially expanded configuration shown in FIG. 2A than in the radially contracted configuration shown in FIG. 1.

As shown in FIG. 2A, the first injection port 141 may be disposed on the radial inside surface of the first expansion member 121. The first injection port 141 may be positioned at the same longitudinal position as the expansion element 120 and, more specifically, it may be positioned at the longitudinal centre of the first expansion member 121. Similarly, the second injection port 142 may be disposed on the radial inside surface of the second expansion member 122. The second injection port 142 may also be positioned at the same longitudinal position as the expansion element 120 and, specifically, at the longitudinal centre of the second expansion member 122. By having the injection ports 141, 142 positioned at the same longitudinal position as the expansion element 120, the adhesive is injected at the point where the vessel is most flattened by the expansion element 120. This therefore results in more effective sealing of the vessel and reduces the required amount of adhesive.

FIG. 2B shows a cross-sectional view of the medical device 100 of FIG. 2A along line BB. FIG. 2B shows how the inner shaft 112 may be positioned within the outer shaft 111. The first injection lumen 143 and the second injection lumen 142 may be positioned within the outer shaft 111.

The suction lumen 131 may be positioned within the inner shaft 111 and connected to the suction port 130 at the distal end of the inner shaft 111.

FIG. 2C shows a cross-section view of the medical device 100 of FIG. 2A along line CC. FIG. 2C shows the inner shaft 112 with the suction lumen 131. The first expansion member 121 and the second expansion member 122 are positioned around the inner shaft 112 on opposite sides. When the first and second expansion members 121, 122 move between the radially contracted configuration and the radially expanded configuration, they hence move in a plane coplanar with the longitudinal axis L of the catheter shaft 110.

The first expansion member 121 may be a hollow tube comprising the first injection lumen 143. Similarly, the second expansion member 122 may be a hollow tube comprising the second injection lumen 144. FIG. 2C further shows how the first injection port 141 may be positioned on the radial inside of the first expansion member 121 and fluidly connected to the first injection lumen 143. Similarly, the second injection port 142 may be positioned on the radial inside of the second expansion member 122 and fluidly connected to the second injection lumen 144. The first injection port 141 and the second injection port 142 may be in the form of a hole or a nozzle, for example, to allow the adhesive to be injected into the vessel from the first and second injection lumen 143, 144.

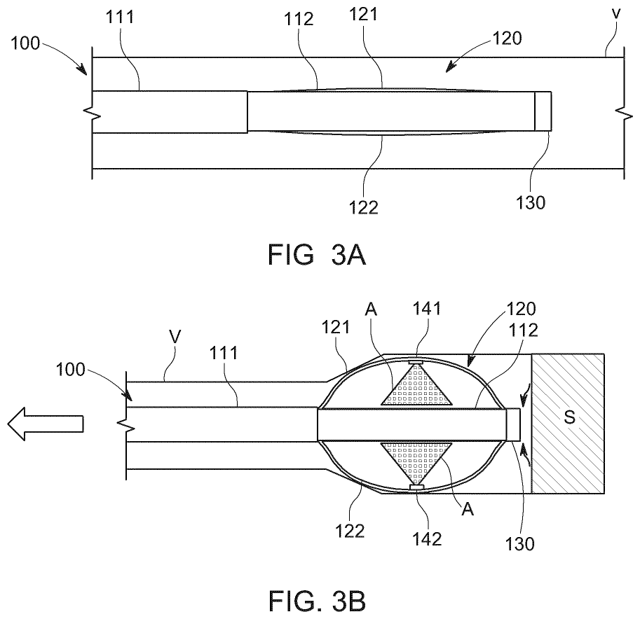

FIGS. 3A and 3B illustrate a method of using the medical device 100 in order to close and seal a vein V. The vein V may be a varicose vein, for example, caused by CVI. The vein V may have damaged valves such that the blood is not effectively transported through vein V and leaks backwards. By closing or sealing vein V, the blood will be rerouted to other healthy veins which can transport blood more effectively.

Firstly, the medical device is inserted into the vein V through an access site. As shown in FIG. 3A, the medical device 100 is then advanced through the vein V to a treatment site where the vein V is to be closed and sealed. During insertion and advancement of medical device 100, the expansion element 120 may be in the radially contracted configuration to allow the medical device 100 to easily be inserted into and advanced through the vein V. The medical device 100 may further be disposed within a sheath (not shown) during the insertion and advancement steps. During insertion and advancement of the medical device 100, the suction lumen 131 may also be used as a guidewire lumen for accommodating a guidewire or a dilator lumen for accommodating a dilator and a guidewire.

Once the medical device 100 is positioned at the treatment site, the expansion element 120 is moved from the radially contracted configuration to the radially expanded configuration, for example, by moving the expansion control element 151 proximally. This causes the first and second expansion members 121, 122 to expand radially outwards and contact the venous wall. As the first and second expansion members 121, 122 are positioned on opposite sides of the catheter shaft 110 and move in a plane coplanar with the longitudinal axis L, this will result in the vein V becoming flattened.

In order to close the vein V and seal it, the vacuum pump 160 and the adhesive pump 170 are turned on and the medical device 100 is pulled proximally, as shown in FIG. 3B. Adhesive A is then injected into the flattened vein V via the first injection port 141 and the second injection port 142 positioned on the first expandable member 121 and the second expandable member 122, respectively. The suction port 130 reduces the pressure in the vein V which aspirates blood from the vein V and further flattens the vein V. The combination of the expansion element 120 and the suction port 130 helps to collapse the vein V in a predictable manner and avoids the need for manual compression of the vein V. Any excess adhesive A is also removed via suction port 130, thereby minimizing the amount of adhesive required to seal and close the vein V. As the medical device 100 is pulled proximally, the vein V is sealed and closed with adhesive A, as shown by sealed vessel section S.

Furthermore, by pulling the medical device 100 proximally, the roughened surface of the expandable members 121, 122 may scrape the walls of the vein V and thereby mechanically irritate the walls of the vein V, and specifically the endothelium and the tunica media layer of cells in the venous walls. This causes fibrosis and scarring of the venous wall thereby encouraging vein closure and helping to effectively seal and close the vein V.

Once the desired length of the vein V has been sealed shut, the vacuum pump 160 and the adhesive pump 170 may be turned off. The expansion element 120 may then be moved from the radially expanded configuration to the radially contracted configuration and removed from the vein V.

As the vein V is closed and sealed, blood will no longer be able to flow through vein V and will be rerouted to other healthy veins which can transport the blood more effectively.

The medical device 100 therefore allows the closure of a vessel, such as a vein, in a safe, quick and effective manner using non-tumescent, non-thermal and non-sclerosant means and avoids the requirement for manual vein compression.

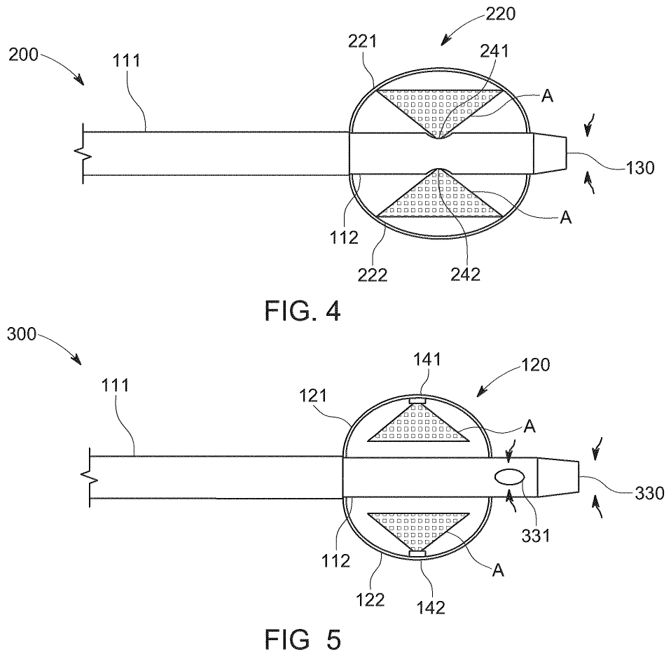

FIG. 4 shows an alternative embodiment of a medical device 200 for closing a vessel such as a vein. The medical device 200 is similar to medical device 100 and features which are the same across the embodiments have been labelled with the same reference numerals.

Medical device 200 differs from medical device 100 in that it comprises a first injection port 241 and a second injection port 242 disposed on the catheter shaft 110, specifically on the inner shaft 112, rather than the first and second expansion members 221, 222. An injection lumen (not shown) may run through the inner catheter shaft 112 to connect the first and second injection ports 241, 242 to the adhesive pump 160. The first and second injection ports 241, 242 may be positioned on opposite sides of the inner shaft 112 and may be positioned in the same plane as the first and second expansion members 221, 222 to allow for effective injection of the adhesive A when the vessel is flattened.

The first and second expansion members 221, 222 may be wires or ribbons, rather than tubes, as they do not need to have an injection lumen running through them. This may simplify the manufacture and assembly of the medical device 200.

The method of using the medical device 200 to close and seal a vein V is similar to the method explained in relation to FIGS. 3A and 3B above, except that the adhesive A is injected through the first and second injection ports 241, 242 on the inner shaft 112, rather than on the first and second expansion members.

FIG. 5 shows an alternative embodiment of a medical device 300 for closing a vessel such as a vein. The medical device 300 is similar to medical device 100 and features which are the same across the embodiments have been labelled with the same reference numerals.

Medical device 300 differs from medical device 100 in that it comprises, in addition to a a first suction port 330 positioned at a distal end of the inner shaft 112, a second suction port 331 positioned proximally of the first suction port 330 and distally of the expansion element 120 and the injection ports 141, 142. The second suction port 331 may be in the form of side holes in the inner catheter shaft 112 and may comprise a first hole and a second hole on opposite sides of the inner shaft 122. The first hole and second hole may be disposed in a plane perpendicular to the plane of the first and second expansion members 121, 122. The addition of the second suction port 331 may ensure a more uniform reduction of the pressure in the vessel and help to more effectively flatten the vessel thus ensuring effective application of the adhesive to seal the vessel.

The method of using the medical device 300 in order to close and seal a vein V is identical to that explained above in relation to FIG. 3A and B.

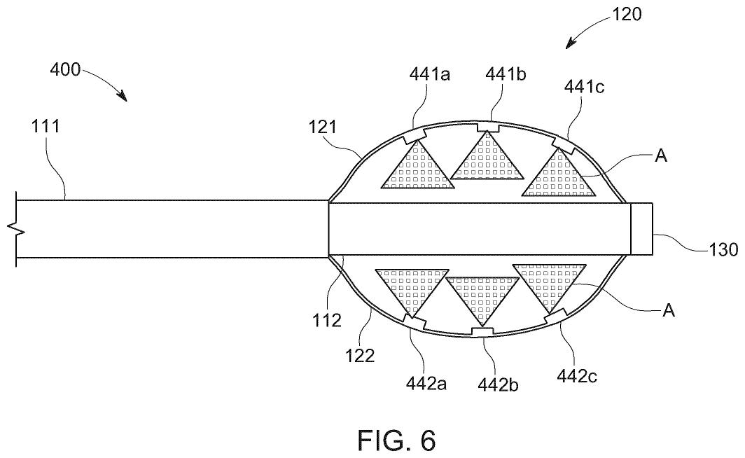

FIG. 6 shows an alternative embodiment of a medical device 400 for closing a vessel such as a vein. The medical device 400 is similar to medical device 100 and feature which are the same across the embodiments have been labelled with the same reference numerals.

The medical device 400 differs from medical device 100 in that each of the first and second expansion members 121, 122 comprises a plurality of injection ports disposed longitudinally along the expansion member. For example, the first expansion member 121 comprises three injection ports 441a, 441b and 441c. Similarly, the second expansion member 122 comprises three injection ports 442a, 442b and 442c.

The plurality of injection ports may help to distribute the adhesive A more uniformly in the vessel, thereby resulting in more effective sealing and closure of the vessel. The size of the injection ports may vary. For example, proximal injection ports may be smaller whilst distal injection ports may be larger, i.e., injection port 441c may be larger than injection port 441b which may be larger than injection port 441a. Similarly, injection port 442c may be larger than injection port 442b which may be larger than injection port 442a.

The method of using the medical device 400 in order to close and seal a vein V is the same as explained above in relation to FIG. 3A and B, except that the adhesive A is injected through the plurality of injection ports 441a-c and 442a-c at the same time.

Various modifications will be apparent to those skilled in the art.

The medical device 100, 200, 300, 400 may not comprise a suction port 130, 330.

The suction port 130 may not be positioned on the catheter shaft 110 but may be positioned on the expansion element 120, for example.

The suction port 130 may not be positioned distally of the expansion element 120 but may be positioned, for example, at the same longitudinal position or proximally of the expansion element.

The suction port 130 may not be positioned distally of the injection ports 141, 142 but rather may be positioned, for example, at the same longitudinal position or proximally of the injection ports 141, 142.

The medical devices 100, 200, 300, 400 may have any suitable number of injection ports.

The injection ports may be positioned on the catheter shaft 110, the expansion element 120, or a combination of both.

The injection ports may not be positioned at the same longitudinal position as the expansion element 120. Rather, the injection ports may be positioned proximally or distally of the expansion element 120, for example.

The injection ports may be the same size or may be differing in size.

The expansion element 120 may comprise fewer or more than two expansion members.

The expansion members may take any suitable form, such as, for example, tubes, wires, struts or balloons.

The expansion members may not be flexible but may comprise struts connected by hinges, for example.

The means for flattening may take any suitable form such as, for example, tubes, wires, struts or balloons.

The first expansion member 121, 221 and the second expansion member 122, 222 may not have a rough surface but may have a smooth surface instead. Alternatively, the first expansion member 121, 221 and the second expansion member 122, 222 may have a serrated surface.

The catheter shaft 110 may not comprise an outer shaft 111 and an inner shaft 112. Rather, the catheter shaft 110 may only comprise an outer shaft and the expansion element 120 may be moved between the radially contracted configuration and the radially expanded configuration with a pull wire or a shape memory material, for example.

The medical device may not comprise a handle 150.

The expansion control mechanism 151 is not limited to a slider but may take any other suitable form, for example, a rotating knob, a moveable rod, or a button.

The medical device may further comprise a guidewire lumen for accommodating a guidewire.

All of the above are fully within the scope of the present disclosure and are considered to form the basis for alternative embodiments in which one or more combinations of the above-described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A medical device for closing a vessel, the medical device comprising:
a catheter shaft defining a longitudinal axis;
an expansion element positioned at or near a distal end of the catheter shaft, the expansion element having a radially contracted configuration and a radially expanded configuration, wherein the expansion element is configured to flatten the vessel in the radially expanded configuration; and
one or more injection ports for injecting an adhesive into the vessel.

2. The medical device of claim 1, further comprising one or more suction ports for reducing the pressure in the vessel.

3. The medical device of claim 2, wherein the one or more suction ports are positioned on the catheter shaft.

4. The medical device of claim 2, wherein at least one of the one or more suction ports is positioned at the distal end of the catheter shaft.

5. The medical device of claim 2, wherein the one or more suction ports are positioned distally of the expansion element.

6. The medical device of claim 2, wherein the one or more suction ports are positioned distally of the one or more injection ports.

7. The medical device of claim 2, further comprising a vacuum pump connected to the one or more suction ports through a suction lumen.

8. The medical device of claim 1, wherein the one or more injection ports are positioned at the same longitudinal position as the expansion element.

9. The medical device of claim 1, wherein at least one of the one or more injection ports is positioned on the catheter shaft.

10. The medical device of claim 1, comprising a plurality of injection ports arranged along the longitudinal axis.

11. The medical device of claim 10, wherein a distal injection port of the plurality of injection ports has a greater opening than a proximal injection port of the plurality of injection ports.

12. The medical device of claim 1, wherein the catheter shaft comprises an injection lumen for supplying an adhesive to the one or more injection ports.

13. The medical device of claim 12, further comprising an adhesive pump connected to the one or more injection ports via the injection lumen.

14. The medical device of claim 1, wherein the expansion element moves between the radially contracted configuration and the radially expanded configuration in a plane coplanar with the longitudinal axis.

15. The medical device of claim 1, wherein the expansion element comprises a first expansion member and a second expansion member positioned on opposite sides of the catheter shaft.

16. The medical device of claim 15, wherein the first expansion member and the second expansion member have a rough or serrated surface.

17. The medical device of claim 15, wherein the first expansion member and the second expansion member are flexible and configured to bend radially outwards in the radially expanded configuration.

18. The medical device of claim 15, wherein the first expansion member and the second expansion member have a convex shape in the radially expanded configuration.

19. The medical device of claim 15, wherein the first expansion member comprises a first tube and the second expansion member comprises a second tube.

20. The medical device of claim 19, wherein at least one of the one or more injection ports is positioned on the first tube and/or the second tube.

21. The medical device of claim 20, wherein the at least one of the one or more injection ports are positioned on the radial inside surface of the first tube and/or the second tube.

22. The medical device of claim 15, wherein the first expansion member comprises a wire or ribbon and the second expansion member comprises a wire or ribbon.

23. The medical device of claim 22, wherein the one or more injection ports are positioned on the catheter shaft.

24. The medical device of claim 1, wherein the expansion element is made from a metal or metal alloy.

25. The medical device of claim 1, wherein the expansion element is made from a superelastic material.

26. The medical device of claim 1, wherein the expansion element is made from a shape-memory material.

27. The medical device of claim 1, wherein the expansion element is made from Nitinol.

28. The medical device of claim 1, wherein the catheter shaft comprises an outer shaft and an inner shaft.

29. The medical device of claim 28, wherein the inner shaft is longitudinally moveable relative to the outer shaft.

30. The medical device of claim 29, wherein a proximal end of the expansion element is connected to the outer shaft and a distal end of the expansion element is connected to the inner shaft such that longitudinal movement of the inner shaft relative to the outer shaft moves the expansion element between the radially contracted configuration and the radially expanded configuration.

31. The medical device of claim 1, wherein the catheter shaft further comprises a guidewire lumen for accommodating a guidewire.

32-36. (canceled)
